(19) United States
(12) Patent Application Publication
(10) Pub. No.: US 2006/0193824 A1
(43) Pub. Date: Aug. 31, 2006

(54) Methods for the treatment of infectious and inflammatory airway diseases

(76) Inventors: Bruce K. Rubin, Winston-Salem, NC (US); Markus O. Henke, Marburg (DE)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/068,141

(22) Filed: Feb. 28, 2005

(51) Int. Cl. A61K 38/19; A61K 31/70; A61K 31/7076; A61K 31/7008; A61K 31/551; A61K 31/519; A61K 31/557; A61K 31/203; A61K 31/202; A61K 31/138

(52) U.S. Cl. 424/85.1; 424/94.64; 514/62; 514/559; 514/560; 514/400; 514/43; 514/220; 514/267; 514/651; 514/573

(57) ABSTRACT

Methods for the treatment of inflammatory and infectious airway diseases are disclosed.

### FIELD OF THE INVENTION

The present invention relates to the fields of pulmonary medicine and protocols for the treatment of infectious and inflammatory airway diseases. More specifically, the invention provides methods for increasing mucin secretion in a patient thereby alleviating the symptoms associated with such disorders.

### BACKGROUND OF THE INVENTION

Several publications and patent documents are cited throughout the specification in order to describe the state of the art to which this invention pertains. Each of these citations is incorporated herein by reference as though set forth in full.

The mucin glycoproteins are the major macromolecular component of mucous gel, which is responsible for the rheologic properties of mucus (D. Thornton, et al., Biochem. J. 276:667-675 (1991)). Gel-forming mucins influence the physical and clearance properties of mucus (B. Rubin and M. King, In Acute Respiratory Failure in Chronic Obstructive Pulmonary Disease. J. Derenne, et al., eds., Marcel Dekker, New York. 391-411 (1996)). Mucus is a protective coating secreted in the healthy airway, whereas sputum is the product of airway inflammation and usually contains cells, inflammatory mediators, bacteria, highly polymerized DNA inflammatory cell necrosis, and mucin polymers (B. Rubin and M. King, In Acute Respiratory Failure in Chronic Obstructive Pulmonary Disease. J. Derenne, et al., eds., Marcel Dekker, New York. 391-411 (1996); T. Boat, et al., Arch. Biochem. Biophys. 177:95-104 (1976)). Chronic airway diseases, such as asthma, chronic bronchitis (CB), and cystic fibrosis (CF), are thought to be associated with mucus hypersecretion. Mucins from the airway mucus of “normal” subjects and from CF sputum have similar molecular mass (10-30×10⁶ D) (D. Thornton, et al., Biochem. J 276:667-675 (1991); D. Thornton, et al., Biochem. J. 265:179-186 (1990)), but mucins are present at higher concentrations (wt/wt) in normal mucus than CF sputum (M. Rose, et al., Pediatr. Res. 22:545-551 (1987)), and DNA is often present at high levels in CF sputum (T. Boat, et al., Arch. Biochem. Biophys. 177:95-104 (1976)).

Respiratory mucins are polydispersed in mass (2-40×10⁶ D) and length (0.5-10 μm) (D. Thornton, et al., Biochem. J. 265:179-186 (1990)) and appear as filaments under electron microscopy. Northern blot and in situ hybridization have shown that at least eight mucin genes (MUC) are expressed as messenger RNA in the respiratory tract (H. Hovenberg, et al., Glycoconj. J. 13:839-847 (1996)). Mucins are classified as secreted or membrane-tethered. Three secreted, gel-forming mucins, MUC2, MUC5AC, and MUC5B, have been reported to be expressed by airway epithelium, but only MUC5AC and MUC5B have been convincingly demonstrated to be major gel-forming mucins in normal or pathologic airway secretions (H. Hovenberg, et al., Biochem. J. 318:319-324(1996); J. Sheehan, et al., Biochem. J. 338:507-513 (1999)). MUC5AC appears to be produced primarily by the goblet cells in the tracheobronchial surface epithelium (H. Hovenberg, et al., Biochem. J. 318:319-324(1996)), whereas MUC5B is secreted primarily by the submucosal glands (C. Wickstrom, et al., Biochem. J. 334:685-693 (1998)).

There are limited published data regarding the specific MUC mucin protein composition of airway secretions in health or disease (H. Hovenberg, et al., Glycoconj. J. 13:839-847 (1996); D. Thornton, et al., Biochem. J. 316:967-975 (1996); S. Kirkham, et al., Biochem. J.361:537-546 (2002)). The current paradigm is that MUC5AC and MUC5B are predominant mucins in airway secretions in normal subjects and in patients with CF, asthma, and CB, and that mucin levels are variable (S. Kirkham, et al., Biochem. J. 361:537-546 (2002)). Although patients with CF have poor mucus clearance and increased airway secretions, there are no published data that demonstrate increased mucin content in the CF airway. Immunohistochemistry studies show that MUC5AC and MUC5B mucins are expressed in the same histologic pattern in CF compared with normal tissues with an increase of MUC5AC-positive cells due to goblet cell hyperplasia and metaplasia in CF tissues (D. Groneberg, et al., Respir. Med. 96:81-86 (2002))). The marked remodeling in the CF airway suggests that with increased secretions, there may be increased mucin content.

There are several steps in the complex process of mucin synthesis, storage, and secretion in which the CF transmembrane ion regulator protein (CFTR) defect could decrease the mucin content of airway secretions. These observations prompted the hypothesis that CF lung epithelial cells may fail to fully differentiate as a result of chronic inflammation, an increased cell turnover or a defective CFTR leading to a decreased secretion of large oligomeric mucin.

It is possible that increased proliferation of the airway epithelium, resulting directly from CF transmembrane ion regulator protein (CFTR) abnormalities or indirectly from infection, injury, and repair, could lead to a generally less mature (differentiated) epithelium and that this, in turn, might result in decreased mucin biosynthesis and secretion. CFTR plays a crucial role in the differentiation of the respiratory epithelium during ovine (F. Broackes-Carter, et al., Hum. Mol. Genet. 11:125-131 (2002)) and murine (J. Larson, et al., Am. J. Physiol. Lung Cell. Mol. Physiol. 279:L333-L341 (2000)) fetal lung development, and may play a regulatory role in the development of the secretory epithelium (J. Larson, et al., Am. J. Physiol. Lung Cell. Mol. Physiol. 279:L333-L341 (2000)). Differences in intracellular pH might enhance proliferation of CFTR-^{-/-} cells. Cytosolic pH is thought to be one of the factors that control the rate of proliferation (J. Barasch, and Q. Al Awqati, J. Cell Sci. Suppl. 17:229-233 (1993)), with cytoplasmatic alkalanization constituting a signal for mitogenesis (A. Elgavish, Biochem. Biophys. Res. Commun. 180:342-348 (1991)). Thus, a mutation in the CFTR may result in an elevated intracellular pH, which stimulates the cell to undergo proliferation more readily and thus increase the rate at which epithelial cells proliferate and differentiate.

A defect in Golgi pH in CF cells may also affect mucin biosynthesis, assembly, and secretion (J. Barasch, et al., Nature 352:70-73 (1991)). Barasch and colleagues suggested that a defect in Golgi pH in CF cells would decrease the activity of pH-sensitive enzymes, which leads to a defect of intracellular glycoprotein processing (J. Barasch, , and Q. Al Awqati, J. Cell Sci. Suppl. 17:229-233 (1993); J. Barasch, et al., Nature 352:70-73 (1991)). Intracellular mucins have a broad distribution of molecular mass (2×10⁶-15×10⁶ D). Analogous to the multisubunit glycoprotein, von Willebrand factor, it can be speculated that mucins are slowly polymerized within storage granules. There is evidence that while in these granules, the molecules can further be oligomerized to form large linear complexes with a relative molecular mass in excess of 10-40×10⁶ D (J. Sheehan, Biochem. Soc. Trans. 23:819-821 (1995)). Altered secretion of the mucin oligomers has also been observed in vitro. Extracellular ATP rapidly increased mucin protein secretion by normal pancreatic cell lines but was not able to induce mucin secretion by the corresponding CF cell line, suggesting that CFTR reduces ATP dependent mucin secretion (C. Montserrat, et al., FEBS Lett. 393:264-268 (1996)). There may also be transcriptional regulation of mucin genes. MUC5AC gene expression was shown to be decreased relative to MUC2 messenger RNA in patients with CF compared with normal control subjects (J. Voynow, et al., Lung 176:345-354 (1998)).

While mucus plugging and chronic expectoration of sputum is present in persons with CF, mucin does not appear to be a major component of these secretions. At least during periods of relative clinical stability, there appears to be more DNA in CF secretions than mucin. The CF protein, CFTR, acts as a cAMP regulated chloride channel (J. Riordan, Annu. Rev. Physiol. 55:609-630 (1993)), but it is not clear how CFTR dysfunction leads to the clinical manifestations of CF lung disease. It may be that mucin secretion is linked to Cl⁻ and water secretion in the airway, increased turnover in secretory cells, or that decreased mucin secretion might be another manifestation of the CFTR defect.

Another possibility is that decreased mucin secretion increases susceptibility to airway infection in the CF airway. Pseudomonas aeruginosa has been shown to bind to airway mucin (R. Ramphal, and S. K. Arora, Glycoconj. J. 18:709-713 (2001)). The dense mucin polymer network appears to be a barrier to bacterial attachment to the epithelium, and thus adequate mucin secretion is probably critical for the initial clearance of airway bacteria. Speculating beyond this, the mucin gel may inhibit bacterial communication, either by binding virulence factors and quorum sensing proteins or by impeding their diffusion to adjacent organisms. Thus, the mucin gel may inhibit the formation of the bacterial biofilms characteristic of CF airway disease.

### SUMMARY OF THE INVENTION

In accordance with the present invention, methods are provided for the treatment of infectious and inflammatory airway diseases. An exemplary method of the invention entails administering a mucin release stimulant to a patient in an amount effective to treat said airway disease. Airway diseases that can be treated with the methods of the invention include chronic bronchitis (CB), cystic fibrosis (CF), bronchiectasis, chronic occlusive pulmonary disease (COPD) and diffuse panbronchiolitis.

Mucin release stimulants contemplated for use in the methods described herein include for example, N-acetyl-glucosamine, arachidonic acid, monohydroxy-eicosatetraenoic acid, prostaglandin thiorphan, leucine-thiorphan, phosphoramidon, P2Y2 purinoceptor agonists, tricyclic nucleotide purinoceptor agonists, cholinergic (M3) agonists (e.g., methacholine, bethanechol, carbachol), adrenergic agonists e.g., isoproterenol, beta 2 agonists e.g., albuterol, epidermal growth factor analogs, tumor necrosis factor alpha, IL-1α, IL-1β, IL-4, IL-10, perflubron, retinoic acid, human neutrophil elastase, histamine and agonists of guanylate cyclase. Also suitable for use in the methods of the invention are agents which stimulate the secretion of endogenously produced mucins.

Preferred mucin release stimulants are P2Y2 purinoceptor agonists, M3 agonists and nucleotriphosphate analogs. The mucin release stimulant(s) may be administered via a variety of routes which include, without limitation, inhalation administration, parenteral administration, intranasal administration, intrapulmonary administration, oral administration, topical administration, intravenous administration, transdermal administration, intra bronchial installation (e.g., by bronchoscope), rectal administration, or subcutaneous administration. A preferred means of administration comprises aerosolized delivery through nebulization, pressurized metered dose inhalers and/or dry powder inhalers.

In yet another embodiment of the invention, mucins are delivered directly to the diseased airway. In a preferred embodiment MUC5AC and/or MUC5AB are delivered. Mucins may be lyophilized and delivered to the diseased airway via a nebulizer. Alternatively, they may incorporated into liposomes prior to delivery.

The methods of the invention should be effective to decrease pulmonary infection and alleviate the symptoms of infectious or inflammatory airway disease. In a preferred embodiment, the methods are effective to increase the amount of MUC5AC and/or MUC5AB in the lungs of said subject, thereby conferring a therapeutic benefit on said subject.

### BRIEF DESCRIPTION OF THE DRAWINGS

FIG. 1 is a western blot which demonstrates that mucin secretion in the CF lung is decreased relative to the healthy control lung. ETT samples from normal control subjects (details in the text) and sputum from patients with CF were examined on 1% agarose gel electrophoresis, blotted to nitrocellulose membranes, and probed with affinity-purified MUC5AC and MUC5B antibodies. Equal volumes of sputum were loaded in each lane and all blots were incubated and exposed together under equal conditions.

FIG. 2 is a graph of the data provided in FIG. 1. Serial diluted dot-blot analysis of ETT (dotted bars) mucus from normal control subjects and sputum from patients with CF (hatched bars). The samples were loaded as volume equivalents from the sputum. The blot was probed with affinity-purified antibodies for MUC5AC and MUC5B. The results for MUC5AC and MUC5B cannot be directly compared with one another because antibody affinity is probably different. (*Students t test P_{—} 0.005).

### DETAILED DESCRIPTION OF THE PREFERRED EMBODIMENTS

Clinicians have long been aware that in certain airway diseases, (e.g., CF, chronic bronchitis, COPD, and bronchiectasis) airways are filled with secretions which cause the patient to chronically expectorate. It has been assumed that these secretions consist of mucin. The paradigm for such disorders, as reviewed by Perez-Vilar and Boucher in Free Radic Biol Med. 2004;37:1564-77, suggests that “abnormal” mucin in CF would increase bacterial infection. Surprisingly, there are no data that definitively show that mucin hypersecretion is deleterious.

In contrast to the widely accepted disease model, the present inventors provide data which indicate that the aberrant airway secretions described above are bacterial and inflammatory cell breakdown products (pus) and contain very little mucin, at least in CF patients. This lack of mucin leads to chronic infection and production of pus by interfering with airway defense. Thus, the present invention provides methods effective for alleviating the symptoms of inflammatory and infectious airway diseases via increasing mucin secretions. Such diseases include, without limitation, cystic fibrosis, chronic bronchitis, bronchiectasis, COPD, and diffuse pan bronchiolitis. Suitable mucin release stimulants are described further herein below.

The phrase “mucin release stimulant” as used herein refers to an agent or combination of agents which are effective to promote production and/or secretion of mucin which is the gel forming component of mucus.

The term “treat” as used herein refers to any type of treatment that imparts a benefit to a patient afflicted with a disease, including improvement in the condition of the patient (e.g., in one or more symptoms), delay in the progression of the disease, etc.

The term “pharmaceutically acceptable” as used herein means that the compound or composition is suitable for administration to a subject to achieve the treatments described herein, without undue deleterious side effects in light of the severity of the disease and necessity of the treatment.

The present invention is primarily concerned with the treatment of human subjects, but the invention may also be carried out on animal subjects, particularly mammalian subjects such as mice, rats, dogs, cats, livestock and horses for veterinary purposes, and for drug screening and drug development purposes.

1. Active Compounds.

Active compounds useful for the present invention are, in general, compounds that stimulate the release of mucin, or “mucin release stimulants”. Numerous such compounds are known. Exemplary compounds are set forth below.

Arachidonic acid, metabolic products of arachidonic acid, monohydroxy-eicosatetraenoic acid, and prostaglandins release mucin from human airways and may be utilized as active agents in the present invention. See, e.g., U.S. Pat. No. 4,939,169; U.S. Pat. No. 6,552,081.

N-acetyl-glucosamine is known to stimulate mucin release in the GI tract and may also be utilized as an active agent in the present invention. See, e.g., U.S. Pat. No. 6,046,179.

Thiorphan, leucine-thiorphan and phosphoramidon stimulate mucin release and may be used as active agents in the present invention. See, e.g., U.S. Pat. No. 5,403,585.

P2Y2 purinoceptor and tricyclic nucleotide purinoceptor agonists may also be effective to stimulate mucus release and can be used in the methods described herein. Such agents include, for example, INS365 and can be obtained from Inspire Pharmaceuticals, Inc. Also see e.g., U.S. Pat. Nos. 5,958,897, 6,451,288, 6,555,675, 6,703,376, 6,713,458, 6,818,629 and 6,673,779.

Cholinergic M3 agonists, including without limitation, agents such as methacholine, bethanechol, and carbachol are also effective to stimulate mucin secretion. See e.g., Kishioka C, Respir Physiol. 2001;126:163-71.

Adrenergic agonists, such as isoproterenol and albuterol are also suitable for use in the methods described herein.

Epidermal growth factor or analogs thereof are effective to stimulate mucin production and thus are suitable for use in the methods of the present invention. Exemplary agents of this type include EGF which can be obtained from Sigma. Also see Gray T E, Guzman K, Davis C W, Abdullah L H, Nettesheim P. Am J Respir Cell Mol Biol 1996; 14(1):104-112.

Tumor necrosis factor alpha has been shown to stimulate MUC5AC release in respiratory epithelial cells. See Voynow J A, Young L R, Wang Y, Horger T, Rose M C, Fischer B M. Am J Physiol 1999; 276(5 Pt 1):L835-L843. Accordingly, the use of TNFα is contemplated in the methods described herein.

Interleukin 1 (e.g., IL-1α and IL-1β has been shown to be a mucus secretagogue. See Cohan V L, Scott A L, Dinarello C A, Prendergast R A., Cell Immunol 1991; 136(2):425-434; Gray T, Coakley R, Hirsh A, Thornton D, Kirkham S, Koo J S et al., Am J Physiol Lung Cell Mol Physiol 2003. These cytokines are available from R& D Systems, Minneapolis, Minn.

IL-4 also modulates mucus production and thus may be useful in the presently claimed methods. See Jayawickreme S P, Gray T, Nettesheim P, Eling T., Am J Physiol 1999; 276(4 Pt 1):L596-L603. IL-4 is also obtainable from R & D Systems.

Another cytokine, IL-10 attenuates excessive inflammation and may also be employed in the methods of the invention. See Chmiel J F, Konstan M W, Knesebeck J E, Hilliard J B, Bonfield T L, Dawson D V et al., Am J Respir Crit Care Med 1999; 160(6):2040-2047. IL-10 is obtainable from R & D systems. Also see U.S. Pat. Nos. 5,833,976, 6,387,364.

Kishioka C, Dorighi M P, and Rubin B K have shown that perfluorooctyl bromide (perflubron) stimulates mucin secretion in the ferret trachea. See Chest 1999; 115(3):823-828. Thus, this reagent should be useful in the practice of the invention.

Koo J S, Yoon J H, Gray T, Norford D, Jetten A M, Nettesheim P have demonstrated that retinoic acid is effective to restore the mucus phenotype in retinoid-deficient human bronchial cell cultures. See Am J Respir Cell Mol Biol 1999; 20:43-52. Retinoic acid is available from Sigma.

Human neutrophil elastase has been shown to increase MUC5AC levels in respiratory epithelial cells. See Voynow J A, Young L R, Wang Y, Horger T, Rose M C, Fischer B M. Am J Physiol 1999; 276(5 Pt 1):L835-L843.

Histamine also stimulates mucous glycoprotein release from human airways and is suitable for use in the present invention. See Shelhamer J H, Marom Z, Kaliner M J Clin Invest 1980; 66:1400-1408.

Further there is evidence that there is a cGMP dependant pathway for stimulating mucin exocytosis, which can be inhibited by a specific inhibitor of soluble guanylate cyclase. See Fischer B M, Rochelle L G, Voynow J A, Akley N J, Adler K B. Am J Respir Cell Mol Biol 1999; 20:413-422. Branka J E. Biochem J 1997; 323 (Pt 2):521-524. This pathway may be stimulated with agents disclosed in U.S. Pat. Nos. 6,291,177 and 6,806,082.

MUC5AC and/or MUC5B may also be delivered directly to the diseased airway to help lubricate and inhibit infection. In one embodiment, the peptides are lyophilized and delivered to the airway via a nebulizer. In yet another embodiment, the therapeutic peptides described herein (e.g., the interleukins and MUC molecules listed above) may be incorporated into liposomes and delivered to the patient lung.

Finally, agents which stimulate the exocytosis of endogenously produced mucins to the surface of the airway are also contemplated for use in the methods of the invention.

When appropriate, certain of the active compounds listed above can be prepared and administered in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

The active compound may be in the form of a pharmaceutically acceptable prodrugs. Such compounds are prodrugs of the compounds of the present invention which are, within the scope of sound medical judgment, suitable for use in contact with the tissues of humans and other animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299 Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

2. Formulations and Administration.

Compositions that are useful in the methods of the invention may be administered systemically in parenteral, systemic, intravenous, oral solid and liquid formulations, intranasal, ophthalmic, suppository, aerosol, topical (e.g., transdermal patch) or other similar formulations. In addition to the appropriate mucin release stimulant, these pharmaceutical compositions may contain pharmaceutically-acceptable carriers and other ingredients known to enhance and facilitate drug administration. Thus such compositions may optionally contain other components, such as adjuvants, e.g., aqueous suspensions of aluminum and magnesium hydroxides, and/or other pharmaceutically acceptable carriers, such as saline. Other possible formulations, such as nanoparticles, liposomes, resealed erythrocytes, and immunologically based systems may also be used to administer the appropriate mucin release stimulant, such as the interleukins, to a patient according to the methods of the invention.

The active compounds disclosed herein may be administered to the lungs of a patient by any suitable means. Preferably, the composition of the invention is administered to the human by a lung inhalation route, i.e., via a nebulizer or other lung inhalation device. Exemplary devices include pressurized metered dose inhalers or dry powder inhalers. In one embodiment, an aerosol suspension of respirable particles comprised of the active compound is inhaled by the subject. The respirable particles may be liquid or solid. See generally, “Therapy for Mucus Clearance Disorders” Rubin, B. K., and van der Shans Crees P. eds. in Lung Biology in Health and Disease, Vol. 188 (2004).

Particles comprised of active compound for practicing the present invention should include particles of respirable size: that is, particles of a size sufficiently small to pass through the mouth and larynx upon inhalation and into the bronchi and alveoli of the lungs. In general, particles ranging from about 1 to 10 microns in size (more particularly, less than about 5 microns in size) are respirable. Particles of non-respirable size which are included in the aerosol tend to deposit in the throat and be swallowed, and the quantity of non-respirable particles in the aerosol is preferably minimized. See, e.g., U.S. Pat. No. 5,292,498 to Boucher.

Liquid pharmaceutical compositions of active compound for producing an aerosol may be prepared by combining the active compound with a suitable vehicle, such as sterile pyrogen free water or phosphate buffered saline.

The dosage of active compound or compounds will vary depending on the condition being treated and the state of the subject, but generally may be an amount sufficient to achieve dissolved concentrations of active compound on the airway surfaces of the subject of from about 10⁻⁷ to about 10⁻³ Moles/liter, and more preferably from about 10⁻⁶ to about 3×10⁻⁴ Moles/liter. Depending upon the solubility of the particular formulation of active compound administered, the daily dose may be divided among one or several unit dose administrations.

Aerosols of liquid particles comprising the active compound may be produced by any suitable means, such as with a pressure-driven aerosol nebulizer or an ultrasonic nebulizer. See U.S. Pat. No. 4,501,729. Additional nebulizers are described in U.S. Pat. Nos. 6,851,626; 6,814,071; 6,679,251; 6,513,727; and 6,513,519.

Aerosols of solid particles comprising the active compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator comprises a chamber having a rotor mounted therein, which rotor carries a gelatin capsule containing a metered dose of dry particle medicament. In use the capsule is pierced, a patient inhales through the chamber, and the rotor is caused to spin at a speed sufficient to dispense the medicament to thereby form an aerosol of dry particles. A second type of illustrative aerosol generator comprises a pressurized canister containing dry particle medicament in a propellant. The propellant is discharged through a metering valve configured to dispense a metered dose of the dry particle medicament into the atmosphere. The propellant evaporates, leaving an aerosol of dry particle medicament. The aerosol, whether formed from solid or liquid particles, may be produced by the aerosol generator at a rate of from about 10 to 150 liters per minute, more preferably from about 30 to 150 liters per minute, and most preferably about 60 liters per minute. Aerosols containing greater amounts of medicament may be administered more rapidly. Additional articles relating to aerosol based delivery methods are provided herein below. See for example, Rubin B K. Inhaled corticosteroids: devices and deposition. Paediatr Respir Rev. 2004;5:S103-06; Rubin B K, Fink J B. The delivery of inhaled medication to the young child. Pediatr Clin North Am. 2003;50:717-31; Rubin B K. Nebulizer therapy for children: the device-patient interface. Respir Care. 2002;47:1314-19 and Rubin B K, Fink J B. Aerosol therapy for children. Respir Care Clin N Am. 2001;7:175-213.

The following example is provided to illustrate an embodiment of the invention. It is not intended to limit the invention in any way

### EXAMPLE 1

### Increasing Mucin Secretion is Effective to Reduce Pulmonary Infection

Patients with CF and CB have extensive mucus plugging in their airways leading to increased susceptibility to infection and decreased pulmonary function. Although it has been speculated that in CF this is due to mucus hypersecretion, there are no published data supporting this hypothesis. Therefore we wished to determine if there is an altered mucin concentration in CF airway secretions by measuring mucin in expectorated sputum.

To test the general hypothesis that CF mucus contains increased mucin content, we examined sputa from patients with CF and those with CB by confocal microscopy and studied CF sputum and mucus from normal subjects by gel electrophoresis and dotblot using specific antibodies to differentiate between the major gel-forming mucins MUC5AC and MUC5B.

The following materials and methods are provided to facilitate the practice of Example 1.

### Collection of Sputum and Mucus

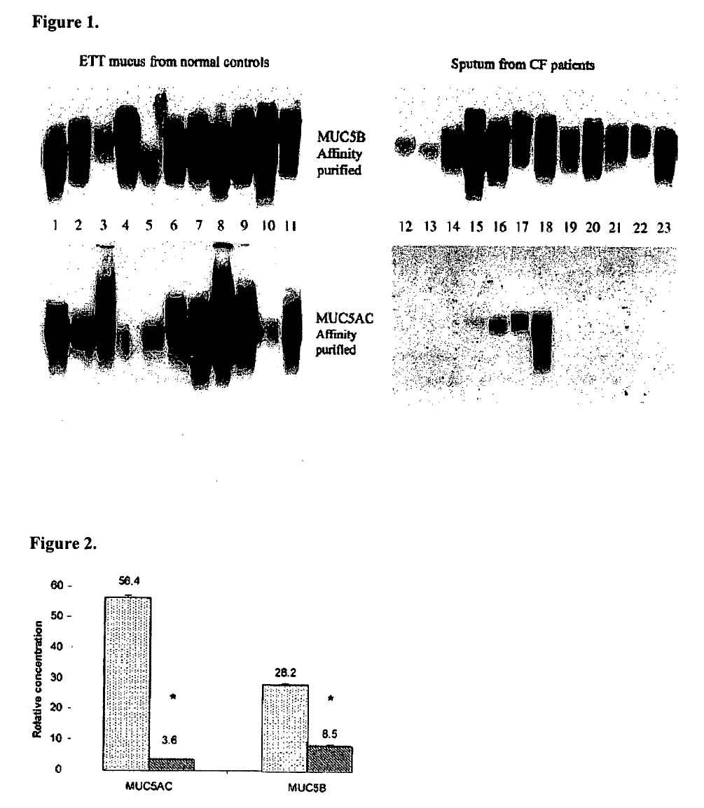

Sputum was collected from patients with CF (n=15) and from patients with bronchitis (n=2) who routinely attend the Wake Forest University clinics. Sputum was collected over 30 min during pulmonary function testing by direct expectoration into a sterile cup. Salivary contamination was minimized by having the subject swallow saliva before expectorating and by separating sputum from saliva by visual inspection at the time of transfer into cryovials for preservation at −70° C. until further processing. The clinical characteristics and demographics of the subjects with CF are given in Table 1. The subject identification numbers given in Table 1 are consistent with the numbering used in FIG. 1.

Mucus was collected from the end of noncuffed endotracheal tubes (ETT) of 11 subjects who had no lung disease but required nonthoracic surgery under general anesthesia. At the time the subject was extubated, the ETT was removed from the airway and mucus coating the tube was removed by scraping the ETT (B. Rubin, et al., Chest 98:101-104 (1990); B. Rubin, et al., Am. Rev. Respir. Dis. 141:1040-1043 (1990)). Sputum and ETT mucus collection were approved by the Wake Forest University Institutional Review Board.

TABLE 1
Clinical characteristics of CF subjects

| Lane | Age | Height | Weight | | | | |
|---|---|---|---|---|---|---|---|
| No. | (Yr) | (Percentile) | (Percentile) | Sex | FVC % | FEV₁ % | Genotype |
| 12 | 9 | 50 | 25 | Female | 49 | 32 | Homozygous dF508 |
| 13 | 10 | 50 | 25 | Female | 22 | 19 | Homozygous dF508 |
| 14 | 21 | 1 | 1 | Male | 36 | 19 | Homozygous dF508 |
| 15 | 18 | 50 | 50 | Male | 88 | 87 | Homozygous dF508 |
| 16 | 10 | 50 | 75 | Female | 83 | 65 | Homozygous dF508 |
| 17 | 19 | 25 | 10 | Female | 98 | 87 | Homozygous dF508 |
| 18 | 31 | 5 | 5 | Male | 56 | 31 | Homozygous dF508 |
| 19 | 31 | 5 | 5 | Male | 57 | 32 | Homozygous dF508 |
| 20 | 36 | 50 | 75 | Female | 73 | 52 | dF508/unknown |
| 21 | 38 | 25 | 5 | Female | 33 | 24 | Homozygous dF508 |
| 22 | 12 | 10 | 5 | Female | 79 | 43 | Homozygous dF508 |
| 23 | 16 | 10 | 1 | Female | 62 | 44 | Homozygous dF508 |

Definition of abbreviations:
FEV₁, forced expiratory volume at 1 second;
FVC, forced vital capacity.

### Confocal Microscopy

Laser scanning confocal microscopy (LSCM) was employed to examine sputum components from three patients with CF and two with CB. Sputa were dual-labeled using 10 μg/ml fluorescent Texas Red-conjugated Ulex europaeus agglutinin (UEA) lectin (Sigma, St. Louis, Mo.) for mucin-like glycoproteins and 1 μM YOYO-1 (Molecular Probes, Eugene, Oreg.) for DNA. A Carl Zeiss LSM 510 (Carl Zeiss, Jena, Germany) and a Leica laser scanning confocal microscope (Leica CLSM; Leica, Lasertechnik GmbH, Heidelberg, Germany) were used to collect images of the stained sputum. Dual excitation wavelengths of 488λ and 568λ were employed to visualize the DNA in combination with mucin. Images were recorded in a planar matrix (X, Y) using the 40× oil objective. Optical sections in the Z-axis were recorded by adjusting the stage height by stepper motors. Quantitative measurements of fluorescence intensity and area were obtained directly from images using VoxelView software (Vital Images, Fairfield, Iowa). Representative fields of interest were visually selected and random coordinates within the field were imaged and analyzed to give mean fluorescent intensities. Serial images for each specimen were analyzed and the mean surface area covered by Texas Red-UEA and YOYO-1 was calculated using NIH Image imaging software (National Institutes of Health, Bethesda, Md.).

### Production of MUC5AC and MUC5B Antibodies

Synthetic peptides with sequences RNQDQQGPFKMC, present in the C-terminal portion and in the two stretches flanking a tandem repeat region of the MUC5AC apoprotein, and RNREQVGKFKMC, present in the cysteine-rich domains of the super-repeats within the central exon of the MUC5B apoprotein, were conjugated to keyhole-limpet hemocyanin (KLH) and used to raise antibodies in rabbits (Genemed Synthesis, San Francisco, Calif.). The antibodies were characterized and specificity was ascertained by preabsorption studies using increasing concentrations of the antigenic peptides as previously done for the original LUM5-1 and LUM5B-2 antibodies (H. Hovenberg, et al., Biochem. J. 318:319-324(1996); C. Wickstrom, et al., Biochem. J. 334:685-693 (1998)). Rabbits were bled four weeks later for antisera, named LUM5-1-WFU for the MUC5AC antibody and LUM5B-2-WFU for the MUC5B antibody. Each antiserum was evaluated for specificity using enzyme-linked immunosorbent assay for the peptides and by Western blot for the proteins in human mucus. To verify the specificity of our antibodies, we performed a polyacrylamide gel electrophoresis with cell lysates, secretions from normal human tracheobronchial epithelial cells (passage 2) (Clonetics Corp., La Jolla, Calif.), and human mucus. The blots were analyzed with antisera for MUC5AC and MUC5B and the preimmune sera of the same rabbit. We found one well-defined band of high molecular weight with the antisera. To increase the specificity of the antibodies and reduce nonspecific binding, we affinity-purified the antipeptide antibody from the whole serum using the immobilized amino acid sequences of interest (SulfoLink-Kit, Pierce Chemical Co., Rockford, Ill.).

### Sputum and ETT Preparation for Gel Electrophoresis and Dot-blot

Protease inhibitors (Protease Inhibitor set 1, Calbiochem, La Jolla, Calif.) were added in equal volumes to the sample during thawing. The samples were homogenized by aspirating several times through progressively smaller needles (final size 28 gauge (G)), diluted 1/10 with phosphate-buffered saline (PBS), and homogenized again with a 28 G needle. Total protein concentration was measured using a BCA-Kit (Pierce).

Sputum and ETT samples were applied in Laemmi buffer (250 mM Tris, pH 6.8; 4% sodium dodecyl sulfate; 20% glycerol; 0.001% bromophenol blue, 20 mM dithiothreitol [DTT]) and electrophoresed in 1% agarose gels (15×15 cm), prepared in running buffer (25 mM Tris, 250 mM glycine, 0.1% sodium dodecyl sulfate). Electrophoresis was performed in a horizontal gel apparatus at 100 V at room temperature. To identify small proteins that remained in the gel, the gel was stopped when the dye front was two-thirds of the distance from the wells. After electrophoresis, proteins were transferred to nitrocellulose membranes by electrical transfer (30V) for 18 h at 4° C.

### Dot-blot

Samples were applied to the top row of a 96-well plate in a potassium thiocyanate (KSCN)-dithiothreitol (DTT) solution (KSCN 0.6 M, DTT 20 mM). The samples were diluted (1:1) from row to row with the KSCN-DTT solution, transferred to a 96-well acrylic Dot-Blot System (Schleicher and Schuell, Dassel, Germany), and blotted to a nitrocellulose membrane while applying a vacuum for 1 min. The blots were then probed with anti-MUC5AC or MUC5B and detected with chemiluminescence to determine the limiting detectable dilution of each sample, reported as the relative concentration. Dot-blots were analyzed by counting the number of visible dots per sample, equivalent to the number of serial dilutions required to reach limiting detectable titer. The sum of the dots of each patient sample was used as an exponent of 2 and the mean was used to compare the relative concentrations of mucins among patient samples, using the equation (2^{x1}+2^{x2})/n. For example, for the ETT mucus concentration of MUC5B, four dots were counted for sample 1, two for sample 2, six for sample 3, and five for sample 4. The calculation (2⁴+2²+2⁶+2⁵)/4 was used to determine the mean of 28.2 shown in FIG. 2.

### Probing the Blots

The membranes were blocked with 5% nonfat skimmed milk in PBS for 30 minutes at room temperature. They were incubated with primary antibodies (1:100 MUC5AC; 1:250 MUC5B) for 1 hour in 1% nonfat skimmed milk in PBS, washed 3 times in PBS for 10 min, and incubated with the secondary horse radish peroxidase-labeled goat-anti-rabbit antibody (1:1000) (Jackson-Immuno, West Grove, Pa.) in 1% nonfat skimmed milk in PBS for 1 hour. Finally, they were washed 3 times in PBS for 10 min. Membranes were developed using the Pico-Developer Kit (Pierce). Exposures were taken on X-Omat Blue XB-1 film (Kodak, Rochester, N.Y.) at equal times. The membranes of the gel electrophoresis/dot-blot were first probed with affinity-purified MUC5AC, stripped, and probed again with affinity-purified MUC5B antibody. We then repeated the gel electrophoresis/dot-blot using new samples and the membranes were then probed first with affinity-purified MUC5B, stripped, and then probed with affinity-purified MUC5AC antibody with equivalent results.

### Deglycosylation

The respiratory mucins were electrophoretically separated by size in a 1% agarose gel and immobilized onto a polyvinylidene-fluoride membrane by an 18 h electrical transfer. The membrane was washed in PBS, incubated at 37° C. for 30 min in neuraminidase (in 50 mM sodium acetate, 150 mM sodium chloride, 100 mM calcium chloride, pH 5.5) to remove sialic acid, washed and followed by an incubation in a glass bottle at 4° C. for 15 min with trifluoromethanesulfonic acid to remove core sugars (D. Thornton, et al., Anal. Biochem. 227:162-167 (1995)). The membrane was then thoroughly washed in PBS, blocked, and probed with anti-mucin antibodies.

### RESULTS

### Mucin-like Glycoconjugates and DNA in CF and CB Sputum using LSCM

In order to determine the relative of amount of mucin in secretions from the CF and bronchitis airways, we used LSCM to visualize polymer components of sputum from three patients with CF and two with CB (R. Tomkiewicz, et al., In Cilia, Mucus, and Mucociliary Interactions. G. Baum, et al., eds. Marcel Dekker, New York. 333-341 (1996)). Specimens were dual-labeled using fluorescent Texas Red-conjugated UEA lectin for mucin glycoprotein and YOYO-1 for DNA. When planar images suggested co-localization as a yellow color, optical sections in the Z-axis were recorded by adjusting the stage height by stepper motors. Co-localization was confirmed if dual wavelength emission was detected in adjacent pixels in both planar and Z-axis sections. In all samples evaluated, DNA and mucin polymers appeared discrete using these criteria. CF sputum contained 45% less mucin (P<0.05) and 416% more DNA (P<0.01) than CB sputum by area (data not shown). In addition, YOYO-1 staining of CB sputa suggested that there was greater amount of DNA in intact inflammatory cell nuclei.

Most likely the increased DNA levels in CF sputum is the result of polymorphonuclear leukocyte necrosis (T. Boat, et al., Arch. Biochem. Biophys. 177:95-104 (1976); R. Picot, et al., Thorax 33:235-242 (1978); M. Lethem, et al., Eur. Respir. J. 3:19-23 (1990)). Thus, the DNA probably has a much greater effect on CF sputum properties and volume than mucins.

### CF Sputum Compared with ETT Mucus Using Gel Electrophoresis and Dot-blot

We measured the gel forming mucin protein content of sputum from patients with CF (n=12) and mucus from normal control subjects (n=11) using gel electrophoresis and dot-blots probed with specific MUC5AC (LUM5-1-WFU) and MUC5B (LUM5B-2-WFU) antibodies. The CF samples were obtained from patients routinely attending the CF clinic at Wake Forest University. The age of the patients with CF was 20.92±3.04 (mean±SEM) years. Clinical and demographic data are summarized in Table 1. For comparison, control mucus was collected from the endotracheal tube (ETT) of surgical patients with no lung disease (age 18.5±8.03 y). The Western blots were probed with anti-peptide antibodies for both MUC5AC and MUC5B and showed significantly decreased content of both mucins that was most striking for MUC5AC, where the protein was not detectable in many samples even after 5 min of film exposure (FIG. 1). We could only detect MUC5AC in most of the CF sputum after overexposing the membrane with a more sensitive developer (pictures not shown). Even with overexposure, no lower molecular bands could be detected by the MUC5AC and MUC5B antibodies. This suggests that mucin fragmentation was unlikely to be an important problem in interpretation.

Since the oligomerization of mucins results in broad banding of high molecular weight polymers, it was difficult to quantify changes in mucin content. Therefore, we used dot-blot to determine the relative concentration of each mucin. The blots revealed a quantitative decrease in both MUC5AC and MUC5B in CF sputum relative to normal mucus (FIG. 2) consistent with the Western blot results. Although the decrease in CF MUC5B was significant (30% of normal mucus, P<0.005), the decline of MUC5AC was more dramatic (˜7% of normal, P<0.005). In 8 out of 12 patient samples titration with the dot-blots was required in order to detect MUC5AC. Because the avidity of the antibodies for MUC5AC and MUC5B is undoubtedly different, these separate results should not be directly compared.

The membranes of the Western and dot-blot were first probed with affinity-purified MUC5AC, then stripped and probed with affinity-purified MUC5B antibody. We repeated the Westerns and dot-blots using new samples probed first with affinity-purified MUC5B, stripped and then probed with affinity-purified MUC5AC antibody. The results were identical.

All samples were loaded on the gel as volume equivalents from the sputum (FIGS. 1 and 2). Since dilution of the sputa could affect the appearance of the Western blots, samples were also loaded onto a gel as total protein equivalents (not shown). The total protein of the volume equivalent samples was 3,809±1,179 μg/ml in the CF samples and 6,296±1,584 μg/ml in the normal mucus. Total protein was normalized to 1,000 μg/ml diluting the samples with PBS prior to repeating the Westerns. The appearance of MUC5AC and MUC5B in the Western gels was similar in both the volume and protein equivalent loaded blots.

To determine if changes in glycosylation would affect antibody binding, we repeated the gel electrophoresis, deglycosylated the mucin bound to the membrane, and probed the membrane with MUC5AC and MUC5B antibodies. We compared the deglycosylated samples, directly with untreated samples on a different membrane under equivalent antibody and exposure conditions. We found that there was no difference in MUC5AC nor MUC5B comparing the deglycosylated to the native mucins, so the decreased mucin content we observed in CF sputum samples was not a result of changes in glycosylation.

To determine whether the mucins were physically sheared by aspiration through the 28 gauge needles, we aspirated three salivary samples and compared them with native salivary samples in a Western blot. We found no significant differences in the MUC5B content of the sheared and control samples. As expected, MUC5AC could not be detected in any of these samples.

Consistent with the finding of reduced mucin in CF airway sputum, Rose obtained mucus by hypertonic saline induction and showed that mucins were present at lower concentrations in three patients with CF compared with two normal volunteers (M. Rose, et al., Pediatr. Res. 22:545-551 (1987)). It was speculated that this was due to increased proteinase activity in vivo. However, our results show that MUC5AC and MUC5B mucins from patients with CF and normal control subjects have the same apparent size (FIG. 1), thus suggesting that these mucins were not proteolytically digested in our CF samples.

The ratio of MUC5B to MUC5AC in the normal control mucus was 0.5, while in CF sputum the ratio increased to 2.4, reflecting a larger decrease in MUC5AC in the CF samples. Davies and coworkers have also reported an increased ratio of MUC5B to MUC5AC in both CF and CB sputum compared with that from healthy control subjects (J. Davies, et al., Biochem. J. 344:321-330 (1999)). Kirkham and colleagues also found an increase in the relative amount of the MUC5B mucin to MUC5AC mucin in both CF and chronic obstructive pulmonary disease (COPD) expectorated sputum in comparison with asthma mucin (P<0.05), but not in comparison to saline-induced mucus from healthy control subjects (S. Kirkham, et al., Biochem. J. 361:537-546 (2002)).

The foregoing is illustrative of the present invention, and is not to be construed as limiting thereof. The invention is defined by the following claims, with equivalents of the claims to be included therein.

That which is claimed is:

1. A method of treating an airway disease in a subject in need thereof, comprising administering at least one mucin release stimulant to said subject in an amount effective to treat said airway disease.

2. The method of claim 1, wherein said airway disease is selected from the group consisting of chronic bronchitis (CB), cystic fibrosis (CF), chronic occlusive pulmonary disease (COPD), bronchiectasis, diffuse panbronchiolitis, chronic sinusitis and rhinitis.

3. The method of claim 1, wherein said secretion is increased by an amount effective to decrease pulmonary infection in said subject.

4. The method of claim 1, wherein said at least one mucin release stimulant is selected from the group consisting of at least one of N-acetyl-glucosamine, arachidonic acid, monohydroxy-eicosatetraenoic acid, prostaglandin thiorphan, leucine-thiorphan, phosphoramidon, a P2Y2 purinoceptor agonist, a tricyclic nucleotide purinoceptor agonist, methacholine, bethanechol, carbachol, isoproterenol, albuterol, an epidermal growth factor analog, tumor necrosis factor alpha, IL-1α, IL-1β, IL-4, IL-10, perflubron, retinoic acid, human neutrophil elastase, histamine and an agonist of guanylate cyclase.

5. The method of claim 1, wherein said mucin release stimulant is administered via a route selected from the group consisting of inhalation administration, intrapulmonary administration, intranasal administration, parenteral administration, oral administration, topical administration, intravenous administration, topical administration, and transdermal administration.

6. The method of claim 5, wherein said at least one mucin release stimulant is administered via inhalation to airway surfaces of at least one lung of said subject.

7. The method of claim 2, consisting of administration of a P2Y2 purinoceptor agonist and a M3 agonist.

8. The method of claim 1, wherein said stimulant is effective to increase the amount of MUC5AC or MUC5AB in the lungs of said subject.

9. The method of claim 1, wherein said airway disease is cystic fibrosis.

10. The method of claim 1, wherein said airway disease is chronic bronchitis.

11. The method of claim 1, wherein said airway disease is bronchiectasis.

12. The method of claim 1, wherein said mucin release stimulant is an agent which promotes exocytosis of endogenously produced mucin.

13. The method of claim 12, wherein said stimulant increases secretion of MUC5AC or MUC5AB.

14. A method of treating airway disease in a subject in need thereof comprising delivering an effective amount of mucin to said subject, in an amount effective to treat said airway disease.

15. The method of claim 14, wherein said mucin is MUC5AC or MUC5AB.

16. The method of claim 15 wherein said mucins are lyophilized and delivered to the patient airway via a nebulizer.

17. The method of claim 15, wherein said mucins are incorporated into liposomes and delivered to the patient airway via a nebulizer.


SEQUENCE LISTING (2 sequences)

SEQ ID NO: 1 - protein, 12 aa, Artificial Sequence
  Synthetic Sequence
RNQDQQGPFKMC

SEQ ID NO: 2 - protein, 12 aa, Artificial Sequence
  Synthetic Sequence
RNREQVGKFKMC